(19) United States
(12) Patent Application Publication
(10) Pub. No.: US 2009/0326490 A1
(43) Pub. Date: Dec. 31, 2009

(54) FECAL INCONTINENCE COLLECTION DEVICE AND METHOD OF USE

(76) Inventors: Donald Jay McMichael, Roswell, GA (US); Sharon Pamperin, Cumming, GA (US); Valerie Lynn Cansler, Alpharetta, GA (US)

Correspondence Address: KIMBERLY-CLARK WORLDWIDE, INC.;Tara Pohlkotte, 401 NORTH LAKE STREET, NEENAH, WI, 54956, US

(21) Appl. No.: 12/165,471

(22) Filed: Jun. 30, 2008

(51) Int. Cl. A61F 5/44

(52) U.S. Cl. 604/328

(57) ABSTRACT

A fecal collection device has a base with a substantially closed proximal end and a substantially open distal end. The base includes a first balloon positioned about an outer surface of the proximal end and a second balloon positioned next to the first balloon. The device also has a catheter having a lumen which is in fluid communication with the base. The first and second balloons are inflated and deflated in a sequence so that pressure against the colon wall is alternated such that the device via the balloons does not cause continuous pressure to the same area of the colon wall. In some instances, fecal material accumulated between an outer surface of the proximal end and the colon wall is moved upward by at least one balloon such that it flows into the proximal end of the base and is eliminated via the catheter.

### BACKGROUND

Patients with bowel movements that are frequent and who are left unclean even for a short period often suffer from irritation along the anal canal, perianus, and perineum ranging from rashes to ulcers. Further passing of stools with such irritations can inflict pain to an already very ill patient as well as exacerbate an existing unpleasant condition and incurring greater medical expenses. Moreover, if left untreated, continued bowel movements have been medically shown to have an adverse impact on a patient's clinical outcome by increasing the possibility of morbidity and mortality.

In particular, fecal incontinence is a common problem in patients in Intensive Care Units. The three common methods to control fecal incontinence are adult diapers, perianal incontinence pouch, and balloon rectal catheters. Diapers and perianal incontinence pouches ( also known as fecal collection bags) allow compromise of skin health.

The disadvantage of the diaper is that there is no barrier between the feces and skin. The moist, occluded environment encourages abrasion, irritation, and infection. Diapers increase work for the nurses, increase cost of care, and can promote compromised skin integrity. They require frequent monitoring, cleaning, and linen changes resulting in use of costly materials and nursing time.

A frequently used fecal containment method is the fecal collection bag, which is usually attached adjacent a patient's rectum via an adhesive wafer that surrounds the rectum. Many factors, however, can influence effective adhesion of the wafer to the skin surface. Skin tear and abrasion injuries can occur with traumatic removal. Skin moisture, edema, and patient anatomy all can preclude the ability to maintain an effective seal.

Balloon rectal catheters are used, but are frequently high maintenance devices that can cause further complications. The balloon rectal catheter is a high maintenance device because of the need to monitor the effects of a balloon attached to the catheter against the wall of the colon. Excessive pressure may result in irritation or necrosis. In addition, introduction of the catheter through the rectum may cause pain.

Therefore, a fecal incontinence collection device is needed. Such a device would desirably be more effective at preventing fecal leakage against a patient's skin, and would reduce or prevent irritation or necrosis of a balloon against the colon wall. The device would desirably also include features which would assist in it's introduction through the rectum.

### SUMMARY OF THE INVENTION

In response to the difficulties and problems discussed herein, a fecal collection device to provided. The fecal collection device has a base having a substantially closed proximal end and a substantially open distal end. The base includes a first balloon positioned on the proximal end of the base and a second balloon positioned on the proximal end of the base near the first balloon. The fecal collection device also has a catheter formed to include a lumen which is in fluid communication with the base. The catheter includes a first lumen in communication with the first balloon, so that the first balloon is inflatable and deflatable. The catheter also includes a second lumen in communication with the second balloon so that the second balloon is inflatable and deflatable. The first and second balloons are inflated and deflated in an alternating sequence so that pressure against the colon wall is alternated so that the device does not continuously cause a same pressure against a same area of the colon wall.

In another aspect of the invention, a fecal collection device is provided, which has a base having a substantially closed proximal end and a substantially open distal end. The base includes a first balloon positioned about the proximal end and a second balloon positioned near the first balloon and about the proximal end, both balloons positioned next to a patient's the colon wall. The fecal collection device also includes a catheter which has a lumen that is in fluid communication with the base. The catheter has a first lumen which is in communication with the first balloon so that the first balloon is inflatable and deflatable. The catheter also has a second lumen which is in communication with the second balloon so that the second balloon is inflatable and deflatable. The first and second balloons are inflated and deflated in a sequence so that pressure against the colon wall is alternated such that the device does not continuously cause a same pressure to a same area of the colon wall. The fecal material accumulated between an outer surface of the proximal end and the colon wall is moved upward during the sequence so that it flows into the proximal end of the base and is eliminated via the catheter.

### BRIEF DESCRIPTION OF THE DRAWINGS

FIG. 1 is a perspective view of one embodiment of the fecal incontinence collection device of the present invention;

FIG. 2 is side view of the fecal incontinence collection device FIG. 1, showing the first and second balloons inflated;

FIG. 3 is a cross-sectional view of FIG. 2 taken along lines 3-3, showing the device positioned in a patient's lower colon and showing the first balloon inflated and the second balloon deflated;

FIG. 4 is a cross-sectional view similar to FIG. 3, but showing the second balloon inflated, a third balloon inflated, and the first balloon deflated;

FIG. 5 is a side view of an additional portion of the catheter of the fecal incontinence collection, showing a first, second, and third free end having a first, second and third valve, respectively;

FIG. 6 is a side view of a free end of the catheter of FIG. 5, and showing the catheter free end connected to a connector of a collection pouch;

FIG. 7 is a perspective view of a second embodiment of the of the fecal incontinence collection device of the present invention; and

FIG. 8 is a cross-sectional view of FIG. 7, taken along lines 8-8, showing an inflated first balloon positioned on an inner surface of the lower portion of the base of the device and showing a second balloon on the inner surface deflated, the base illustrated without an upper portion's ribs.

### DEFINITIONS

As used herein the following terms have the specified meanings, unless the context demands a different meaning, or a different meaning is expressed; also, the singular generally includes the plural, and the plural generally includes the singular unless otherwise indicated.

As used herein, the terms “comprise”, “comprises”, “comprising” and other derivatives from the root term “comprise” are intended to be open-ended terms that specify the presence of any stated features, elements, integers, steps, or components, but do not preclude the presence or addition of one or more other features, elements, integers, steps, components, or groups thereof. Similarly, the terms “include”, “includes”, “has” and/or “have”, and derivatives thereof, are intended to be interpreted as the word “comprise”, and are intended to be open-ended terms that specify the presence of any stated features, elements, integers, steps, or components, but do not preclude the presence or addition of one or more other features, elements, integers, steps, components, or groups thereof.

As used herein, the term “couple” includes, but is not limited to, joining, connecting, fastening, linking, tying, adhering (via an adhesive), or associating two things integrally or interstitially together.

As used herein, the term “configure” or “configuration”, and derivatives thereof means to design, arrange, set up, or shape with a view to specific applications or uses. For example: a military vehicle that was configured for rough terrain; configured the computer by setting the system's parameters.

As used herein, the terms “substantial” or “substantially” refer to something which is done to a great extent or degree; a significant or great amount; for example, as used herein “substantially” as applied to “substantially” covered means that a thing is at least 70% covered.

As used herein, the term “unitary” refers to a unitary component, i.e., a whole, un-divided, un-separated component formed from one piece of material(s).

As used herein, the term “about” adjacent to a stated number refers to an amount that is plus or minus ten (b 10) percent of the stated number.

These terms may be defined with additional language in the remaining portions of the specification.

### DETAILED DESCRIPTION OF THE INVENTION

Reference will now be made in detail to one or more embodiments of the invention, examples of which are illustrated in the drawings. Each example and embodiment is provided by way of explanation of the invention, and is not meant as a limitation of the invention. For example, features illustrated or described as part of one embodiment may be used with another embodiment to yield still a further embodiment. It is intended that the invention include these and other modifications and variations as coming within the scope and spirit of the invention.

A fecal collection device is used with a patient who is receiving medication, or the like, which maintains a patient's fecal material in a liquid state. It will be understood that such a liquid state is desirable when such a device is utilized.

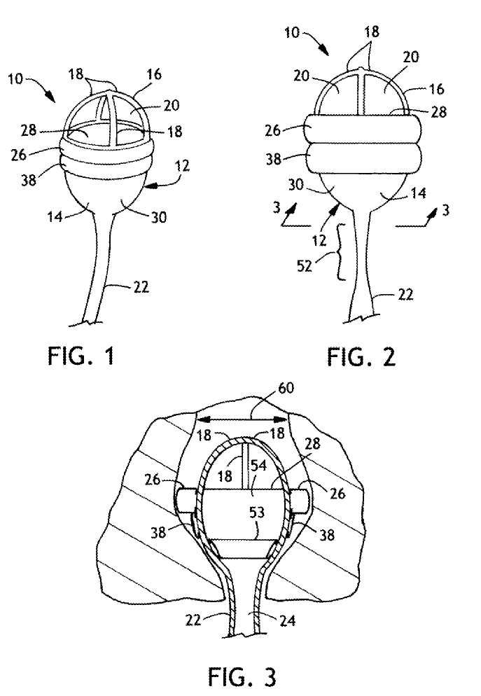

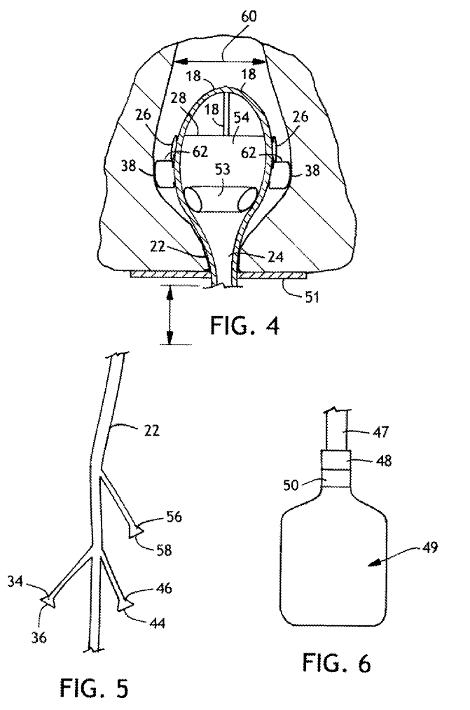

Turning now to the drawings as illustrated in FIGS. 1-8, and in particular, to one embodiment of the present invention shown in FIGS. 1-6, a fecal incontinence collection device 10 is illustrated. The fecal incontinence device 10 may desirably include a generally ovoid-shaped base 12, as illustrated in FIGS. 1 and 4. The base 12 may comprise a solid lower portion 14. The base may also comprise a upper portion 16 having a plurality of ribs 18 which provide a plurality of openings 20 therebetween. The upper portion 16 and the lower portion 14 cooperate to provide the base 12. Desirably, one or more of the openings 20 are large enough to permit colonoscopy to be performed, without the need to remove the device 10. Alternatively, the base may will be provided without the upper portion and its plurality of ribs (not shown). It will be appreciated that the shape of the base 12 or any component thereof is non-limiting, and any shape which operates as shown and/or described herein may be utilized.

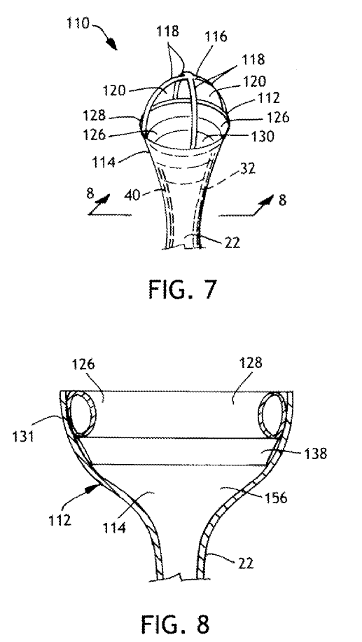

The base 12 is coupled to or desirably integrally formed with a catheter 22. The base 12 and catheter 22 are in fluid communication via a lumen 24 formed therethrough. The base 12 desirably includes a first balloon 26 (FIGS. 1, 2 and 3) positioned about an outer edge 28 of an outer surface 30 of the lower portion 14. The catheter 22 desirably includes a first lumen 32 formed therein or provided therewith which has a valve 34, as illustrated in FIG. 5, provided at a first free end 36 thereof. The first lumen 32 (shown schematically via phantom lines in FIG. 7) desirably has an opening (not shown) formed in the outer surface 30 of the lower portion 14 of the base 12, which is positioned underneath the first balloon 26. Air or liquid, such as sterile water, sterile saline, and so forth, introduced through the valve 34 at the free end 36 travels through the first lumen 32 to inflate the first balloon 26.

Similarly, the base 12 desirably also includes a second balloon 38 (FIGS. 1, 2 and 4) positioned next to the first balloon 26 about the outer edge 28 of the outer surface 30 of the lower portion 14. The catheter 22 desirably also includes a second lumen 40 (shown schematically via phantom lines in FIG. 7) formed therein or provided therewith which has a valve 42 (FIG. 5) provided at a second free end 44 thereof. The second lumen 40 desirably also includes an opening (not shown) formed in the outer surface 30 of the lower portion 14 of the base 12, which is positioned underneath the second balloon 38. Air or liquid, such as sterile saline, introduced through the valve 42 at the second free end 44 travels through the second lumen 40 to inflate the second balloon 38.

The catheter 22 also includes a free end 47 which may include at least one connector 48 which terminates in a collection container or pouch 49, as shown in FIG. 6. Such a pouch 49 desirably also includes a connector 50 which releaseably connects to the connector 38 of the catheter 22. The pouch 49 permits collection of liquid fecal material, and may be emptied and re-used, or, alternatively, disposed of and replaced. An anchoring device (FIG. 4), such as, for example, adhesive wings 51 coupled to the catheter 22, may be used to prevent the device 10 from being pulled out of position by the pouch 49 or movement of a patient.

The fecal collection device 10 may be provided with an introducer (not shown) in order to introduce the base of the device through a patient's rectum. Such an introducer desirably has a have a smooth and relatively narrow outer structure, such as, for example, but not by way of limitation, a hollow cylindrical structure with an opening provided in each end of the structure. The device may be pushed through the structure and the structure inserted at least partially through a patient's rectum and into a patient's lower colon. Once the device is ejected from such a structure, the structure is removed over the catheter (not shown). Alternatively, the structure may have a pair of tabs and be scored such that the structure is easily split into to sections for removal (not shown).

The catheter 22 may include a narrow portion 52 (FIG. 2) having a narrower diameter than portions of the catheter 22 both proximal and distal to the narrow portion 52. The narrow portion 52 is desirably positioned through a patient's rectum.

In yet another alternative, the catheter 22 or the device 10 may include a third balloon 53 (FIG. 4) positioned on an inner surface 54 of the lower portion 14 of the base 12. In this alternative, the third balloon 53 would desirably be used to replicate the natural function of a patient's anal sphincter, and may be used to re-train a patient's body for bowel movements when a patient may have a disorder of the bowel system which is not specific to critical care. The third balloon 53 would be inflated via a third lumen (not shown) formed in or provided with the catheter 22, via a third valve 56 on a third free end 58.

In a method of use of the device 10 of FIGS. 1-6, the first and second balloons 26 and 38 operate to reduce pressure on a patient's colon wall 60, and to prevent accumulation of fecal material adjacent thereto. Pressure on the colon wall and fecal material collected thereagainst may cause irritation, necrosis, and/or infection. That is, desirably, the second balloon 38 may be positioned against or attached at least partially to the first balloon 26. In operation, when the device 10 is positioned in a colon wall 60, the second balloon 38 is inflated. Thereafter, to reduce the pressure by the second balloon 38 on the colon wall 60, and to remove the fecal material that accumulates on an upper portion 62 of the second balloon 38 (FIG. 4), the first balloon 26 is inflated. As the first balloon 26 inflates, it moves upward, thereby moving and lifting fecal material upward (not shown) so that the fecal material flows into the lower portion 14 of the base 12, and is not retained adjacent the colon wall 60 to cause irritation or infection. The second balloon 38 is then deflated via the second valve 42 in the second lumen 40. In this manner, the pressure on a patient's colon wall 60 by the second balloon 38 is removed. And, the fecal material adjacent the device 10 and the colon wall 60 is moved away therefrom and flows into the lower portion 14 of the device 10 and through the catheter 22. It will be appreciated that the first and second balloons 26 and 38 will desirably be inflated alternately, and desirably in the pattern described above, so that fecal material is not held between the first and second balloons 26, 38 and the colon wall 60. Instead, use of this specific pattern moves the fecal material so that it is routinely lifted, moved and eliminated through the catheter 22.

Turning now to the drawings as illustrated in FIGS. 7 and 8, a fecal incontinence collection device 110 similar to the device 10 is illustrated. The fecal incontinence device 110 desirably includes a generally base 112. The base 112 may comprise a solid lower portion 114 which forms, for example, but not by way of limitation, a trumpet-shape. The base 112 may also comprise an upper portion 116 having a plurality of ribs 118 which provide a plurality of openings 120 therebetween. The upper portion 116 and the lower portion 114 cooperate to provide the base 112. Desirably, one or more of the openings 120 are large enough to permit colonoscopy to be performed, without the need to remove the device 110. It will be appreciated that the shape of the base 112 or any component thereof is non-limiting, and any shape which operates as shown and/or described herein may be utilized.

The base 112 is coupled to or desirably integrally formed with the catheter 22. The base 112 and catheter 22 are in fluid communication via a lumen 24 formed therethrough. The base 112 desirably includes a first balloon 126 positioned about an inner edge 128 of an inner surface 130 of the lower portion 114. The catheter 22 desirably includes, as illustrated schematically in FIG. 7, a first lumen 32 formed therein or provided therewith which has a first valve 34 (FIG. 5) provided at a first free end 36 thereof. The first lumen 32 desirably has an opening (not shown) formed in the inner surface 130 of the lower portion 114 of the base 112, which is positioned underneath the first balloon 126. Air, or, alternatively, sterile water, sterile saline, and so forth, introduced through the valve 34 at the free end 36 travels through the first lumen 32 to inflate the first balloon 126.

Similarly, the base 112 desirably also includes a second balloon 138 positioned next to the first balloon 126 about the inner edge 128 of the inner surface 130 of the lower portion 114. The catheter 22 desirably also includes a second lumen 40 formed therein or provided therewith which has a second valve 42 provided at a second free end 44 thereof. The second lumen 40 desirably also includes an opening (not shown) formed in the inner surface 130 of the lower portion 114 of the base 12, which is positioned underneath the second balloon 138. Air, or, alternatively, a liquid, such as sterile saline, introduced through the valve 42 at the second free end 36 travels through the second lumen 40 to inflate the second balloon 138.

The fecal collection device 110 may include any combination of components or all of the components shown and/or described in the first and second embodiments herein, as illustrated in FIGS. 1-8.

In a method of use of the second embodiment of the present invention (FIGS. 7 and 8), the first and second balloons 126 and 138 operate to reduce pressure of an outer surface 131 of the device 110 on a patient's colon wall. The shape of the lower end 114 of the base 112 of the device 110 operates to prevent accumulation of fecal material adjacent thereto. That is, desirably, one balloon, such as the first balloon 126, may be inflated while the other balloon, such as the second balloon 138, may be deflated. In operation, when the device 110 is positioned in a patient's colon, the first balloon 126 is inflated, and the first balloon 126 provides some pressure through the lower end 114 of the base 112 to the colon wall (not shown). Thereafter, to reduce the pressure, the second balloon 138 is inflated while the first balloon 126 is deflated. In this manner, the pressure on a specific area of a patient's colon wall is removed periodically. Therefore, no portion of the colon wall must sustain continuous pressure of the device 110. Such periods of inflation or deflation are desirably determined by health care personnel. Therefore, such periods may be every four (4) hours, every twelve (12) hours, every twenty-four (24) hours, and so forth.

The fecal collection device 10 and 110, and any component(s) thereof, may be formed from any material or combination of materials, so long as the device 10 and 110 operates as shown and/or described herein. Further, any component or group of components described herein may be combined in any manner so long as the device 10 operates as shown and/or described herein.

While the present invention has been described in connection with certain preferred embodiments it is to be understood that the subject matter encompassed by way of the present invention is not to be limited to those specific embodiments. On the contrary, it is intended for the subject matter of the invention to include all alternatives, modifications and equivalents as can be included within the spirit and scope of the following claims.

What is claimed is:

1. A fecal collection device, comprising:
a base having a substantially closed proximal end and a substantially open distal end;
a catheter formed to include a lumen therethrough in fluid communication therewith the base; and
means for alternating pressure against a patient's colon wall such that the device does not continuously cause pressure to a same area of the colon wall.

2. The fecal collection device of claim 1, wherein the means includes a a first balloon positioned on the proximal end of the base and a second balloon positioned near the first balloon on the proximal end of the base.

3. The fecal collection device of claim 2, wherein the catheter further comprises a first lumen in communication with the first balloon such that the first balloon is inflatable and deflatable, and a second lumen in communication with the second balloon such that the second balloon is inflatable and deflatable.

4. The fecal collection device of claim 2, wherein when the device is positioned within a patient's colon, the first balloon is positioned on an outer surface of the proximal wall of the base and next to the patient's colon wall and a second balloon is positioned near the first balloon on an outer surface of the proximal wall of the base and next to the colon wall.

5. The fecal collection device of claim 4, wherein the first and second balloons are inflated and deflated in a sequence so that pressure against the colon wall is alternated such that the balloons do not continuously cause pressure to a same area of the colon wall.

6. The fecal collection device of claim 5, wherein fecal material accumulated above the second balloon and between an outer surface of the proximal end and the colon wall is moved upward when the first balloon is inflated such that it flows into the proximal end of the base and is eliminated via the catheter.

7. The fecal collection device of claim 6, wherein the sequence further comprises the first balloon inflated simultaneously while the second balloon is inflated, so that fecal material between the colon wall and the outer surface of the proximal end is carried upward by the first balloon to permit the fecal material to flow into the proximal end of the device, then the second balloon is deflated.

8. The fecal collection device of claim 1, further comprising a third balloon that, when inflated, substantially blocks the catheter.

9. The fecal collection device of claim 1, further comprising a collection pouch.

10. The fecal collection device of claim 1, wherein the first lumen further comprises a free end having a valve for inflation and deflation of the first balloon, and wherein the second lumen further comprises a free end having a valve for inflation and deflation of the second balloon.

11. A fecal collection device, comprising:
A base having a substantially closed proximal end and a substantially open distal end; the base having a first balloon positioned on the proximal end and a second balloon on the proximal end and positioned near the first balloon; and
a catheter formed to include a lumen therethrough in fluid communication therewith the base, the catheter including a first lumen in communication with the first balloon such that the first balloon is inflatable and deflatable, the catheter including a second lumen in communication with the second balloon such that the second balloon is inflatable and deflatable,
wherein the first and second balloons are inflated and deflated in a sequence so that pressure against the colon wall is alternated such that the device does not continuously cause a same pressure to a same area of the colon wall.

12. The fecal collection device of claim 11, wherein fecal material accumulated on an upper surface of the second balloon when inflated and between an outer surface of the proximal end and the colon wall is moved upward when the first balloon is inflated such that the fecal material flows into the proximal end of the base and is eliminated via the catheter.

13. The fecal collection device of claim 11, wherein the sequence further comprises inflating one balloon, then inflating another balloon, then deflating the one balloon.

14. The fecal collection device of claim 13, wherein the first lumen further comprises a free end having a valve for inflation and deflation of the first balloon, and wherein the second lumen further comprises a free end having a valve for inflation and deflation of the second balloon.

15. The fecal collection device of claim 13, wherein the sequence further comprises the first balloon inflated simultaneously while the second balloon is inflated, so that fecal material between the colon wall and the outer surface of the proximal end is carried upward by the first balloon to permit the fecal material to flow into the proximal end of the base, then the second balloon is deflated.

16. The fecal collection device of claim 1, further comprising a third balloon that, when inflated, substantially blocks the catheter.

17. The fecal collection device of claim 1, further comprising a collection pouch.

18. A fecal collection device, comprising:
A base having a substantially closed proximal end and a substantially open distal end; the base having a first balloon positioned between the proximal end and a patient's colon wall and a second balloon positioned near the first balloon and between the proximal end and the colon wall; and
a catheter formed to include a lumen therethrough in fluid communication therewith the base, the catheter including a first lumen in communication with the first balloon such that the first balloon is inflatable and deflatable, the catheter including a second lumen in communication with the second balloon such that the second balloon is inflatable and deflatable,
wherein the first and second balloons are inflated and deflated in a sequence so that pressure against the colon wall is alternated such that the device does not continuously cause a same pressure to a same area of the colon wall, and fecal material accumulated between an outer surface of the proximal end and the colon wall is moved upward such that it flows into the proximal end of the base and is eliminated via the catheter.

19. The fecal collection device of claim 18, wherein the sequence further comprises the first balloon inflated simultaneously while the second balloon is inflated, so that fecal material on an upper surface of the second balloon and between the colon wall and the outer surface of the proximal end is carried upward by the first balloon to permit the fecal material to flow into the proximal end of the device, then the second balloon is deflated.

20. The fecal collection device of claim 18, further comprising a third balloon that, when inflated, substantially blocks the catheter.

21. The fecal collection device of claim 18, wherein the first lumen further comprises a free end having a valve for inflation and deflation of the first balloon, and wherein the second lumen further comprises a free end having a valve for inflation and deflation of the second balloon.

22. The fecal collection device of claim 18, further comprising a collection pouch.
